(19) United States
(12) Patent Application Publication
(10) Pub. No.: US 2004/0259076 A1
(43) Pub. Date: Dec. 23, 2004

(54) Nano and micro-technology virus detection method and device

(75) Inventor: David Farrow, Toronto (CA)

Correspondence Address: TESTA, HURWITZ & THIBEAULT, LLP, HIGH STREET TOWER, 125 HIGH STREET, BOSTON, MA, 02110, US

(73) Assignee: Accella Scientific, Inc., Toronto (CA)

(21) Appl. No.: 10/601,378

(22) Filed: Jun. 23, 2003

(51) Int. Cl. C12Q 1/70

(52) U.S. Cl. 435/5

(57) ABSTRACT

The invention relates to methods and devices for detecting the presence of a particle of interest (hereinafter an analyte particle) in a fluid. A detection device exemplary of the present invention filters a sample of the fluid to remove particles larger than the analyte particles. A reagent solution, containing reagent particles smaller than the analyte particles, is then added to the sample. The reagent particles will react with the analyte particles, if any are present, to form reagent-analyte complexes which are larger than the analyte particles. The sample is then filtered a second time to remove particles the same size as or smaller than the analyte particles. The sample is then tested for the presence of reagent-analyte complexes to detect the presence of the analyte particle in the fluid.

### FIELD OF THE INVENTION

The present invention relates to methods and devices for detecting the presence of analyte particles, such as viruses, in a biological fluid.

### BACKGROUND OF THE INVENTION

Controlling the spread of infectious diseases is a significant challenge facing society today. In meeting this challenge, effective and efficient methods of virus detection are critical.

While many techniques of virus detection are known and in use, they have several disadvantages. For example, many detection tests must be performed by skilled technicians in laboratories. This increases both the cost of the test and the time it takes to obtain results. Additionally, many detection tests are performed on blood samples, and so typically the blood sample must first be taken by a skilled technician in a laboratory, clinic or hospital setting. Again, this causes the tests to be more expensive and time consuming, as well as, possibly inconvenient for the person being tested.

The necessary involvement of skilled technicians also makes most current tests inappropriate for home testing. For people who have difficulty leaving their homes or who live in remote areas, such tests are inconvenient. Such tests are also undesirable for people who are reluctant to have others know they are being tested for a particular virus. In some cases, a person may be stigmatized for simply being a suspected carrier of a virus. Many people would prefer at home testing to avoid this possibility

Another disadvantage of some known detection devices, is that they must be discarded after a single use. Often, because potentially hazardous biological fluids are involved, special precautions must be taken in their disposal. Again, this may add to the expense of such devices while making them less convenient.

Furthermore, many known tests do not detect the virus itself, instead they detect the antibodies that an infected person's body produces in response to the virus. As a result, there is often a delay after a person becomes infected with a virus before its presence can be detected. For standard human immunodeficiency virus (HIV) tests, which rely on antibody detection, it can take anywhere from three months to a year from the date of infection for a body to produce enough anti-HIV antibodies to test positive.

Accordingly, there is need for an inexpensive, fast and convenient method and device for virus detection.

### SUMMARY OF THE INVENTION

The invention relates to methods and devices for detecting the presence of a particle of interest (hereinafter an analyte particle) in a fluid. A detection device exemplary of the present invention filters a sample of the fluid to remove particles larger than the analyte particles. A reagent solution, containing reagent particles smaller than the analyte particles, is then added to the sample. The reagent particles will react with the analyte particles, if any are present, to form reagent-analyte complexes which are larger than the analyte particles. The sample is then filtered a second time to remove particles the same size as or smaller than the analyte particles. The sample is then tested for the presence of reagent-analyte complexes to detect the presence of the analyte particle in the fluid.

Detection devices exemplary of the present invention can be fabricated using nanotechnology and microtechnolgy techniques. Preferably, devices exemplary of the present invention are hand-held devices suitable for home use. They may be mechanically controlled by a user or electronically controlled by a processing element. Advantageously, devices exemplary of the present invention can be either disposable or reusable

In accordance with an aspect of the present invention, a method for detecting the presence of an analyte particle in a fluid, the method including, sequentially: filtering a sample of the fluid to remove particles in the sample larger than the analyte particle; adding to the sample a reagent that interacts with the analyte particle, to form an analyte particle-reagent complex that is larger than the analyte particle; filtering the sample to remove particles from the sample that are smaller than the analyte particle-renagent complex; testing the sample for the presence of the analyte particle-reagent complex to detect the presence of the analyte particle in the fluid.

In accordance with a further aspect of the present invention, a lab-on-a-chip for detecting the presence of an analyte particle in a fluid, including: a first chamber for receiving a sample of the fluid; a second chamber for receiving a reagent that reacts with the analyte particle in the sample to form an analyte particle-reagent complex, larger than the analyte particle; a filter in flow communication with the second chamber, the filter sized to pass the analyte particle, and block the analyte particle-reagent complex; and a detector for detecting the presence of the analyte particle-reagent complex in the second chamber

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures which illustrate by way of example only, embodiments of this invention:

FIG. 1 is a cross-sectional top view of a virus detection device, exemplary of an embodiment of the present invention;

FIG. 2 is an enlarged schematic view of a portion of the device of FIG. 1 showing the flow of a blood sample;

FIG. 3 illustrates a virus and protein particles reacting to form a virus-protein complex;

FIG. 4 is another enlarged schematic view of a portion of the device of FIG. 1 showing the flow of the blood sample through in the direction, opposite that of the flow shown in FIG. 2; and

FIG. 5 is a cross-sectional top view of a virus detection device, exemplary of another embodiment of the present invention.

### DETAILED DESCRIPTION

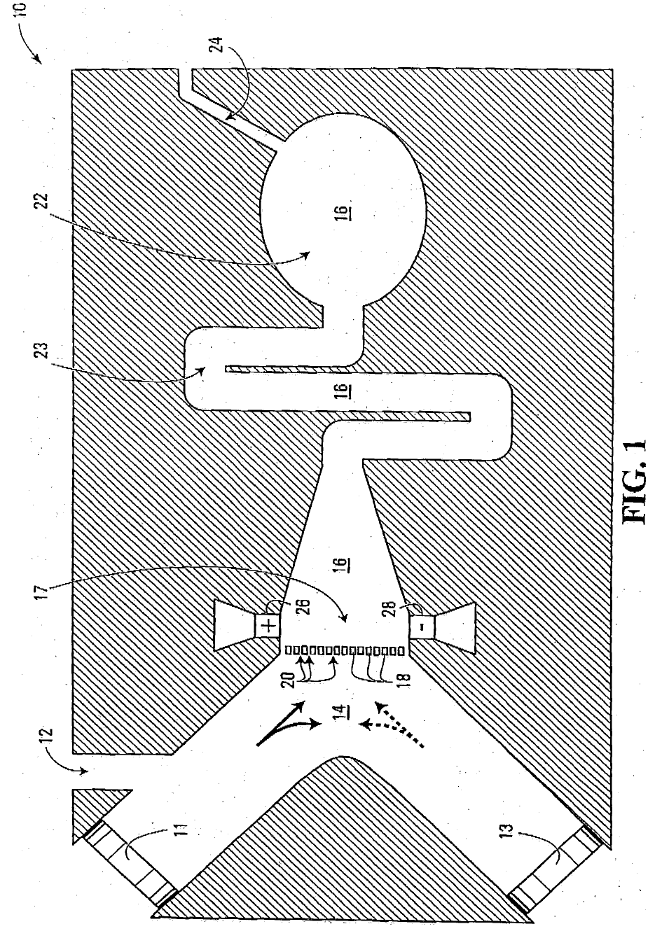

FIG. 1 illustrates a virus detection device 10, exemplary of an embodiment of the present invention. Preferably, the device is a hand-held device known as a “lab-on-a-chip”, manufactured using microtechnology or nanotechnology fabrication methods.

Nanotechnology and microtechnology fabrication methods are generally known in the art. Nanotechnology permits the creation, use, or manipulation of objects at the nanoscale, usually in the 0-01 to 100 nanometer (nm) range. Microtechnology operates similarly at the larger microscale. Nanotechnology and microtechnology manufacturing processes, materials, and devices are used in a wide variety of fields including microelectromechanical systems (known as MEMS), nanomaterials, and microfluidic systems. As micro and nanoscale research advances, it is expected that the sophistication with which such devices can manipulate objects on the nanoscale will grow while the cost of these devices will decrease.

Nanotechnology and microtechnology techniques allow fabrication of “Lab-on-a-chip” devices. “Lab-on-a-chip” devices analyze tiny drops of fluids or chemicals in short periods of time using microfluidic channels. These devices integrate mixing, incubation, separation, detection and data processing in a hand-held device. Such devices may, for example, be fabricated from micro-injection molded plastic. Alternatively, they may be fabricated using LIGA process or any other method known in the art. Embodiments of the present invention exploit “lab-on-a-chip” technology to provide a portable device for virus detection.

In the following description virus detection device 10 is discussed in the context of detecting HIV in a blood sample, however, the device is not so limited. For example, device 10 could be used with biological fluids other than blood, for example, saliva, urine, or embryonic fluid. It could also be used to assay non-biological fluids such as waste water, drinking water, or any other liquid medium containing analyte particles of interest. Analyte particles other than viruses could also be detected, for example, proteins or bacteria,

Device 10 is preferably a hand-held device. The micro-formed components have dimensions on the order of micrometers, as will be described in greater detail below, while the overall dimensions will very depending on the size of the housing.

Device 10 includes an opening 12 for receiving a biological fluid, such as blood; a first fluid chamber 14; and a second fluid chamber 16. Device 10 may also include a one-way exit valve (not shown) to enable air and other gases to escape from the device. Opening 12 leads to first fluid chamber 14. While opening 12 is depicted in FIG. 1 as leading from the side of device 10, alternatively it may lead from the top of device 10 so that the biological fluid would be induced to flow into first fluid chamber 14 by gravity. Opening 12 may also include a one-way valve to prevent fluid backwash.

First fluid chamber 14 is preferably generally V-shaped as shown in FIG. 1. Preferably, the arms of the ‘V’ may have a square cross-section with a width between 20 micrometers and 1 millimeter. They may be several millimeters in length.

Pushing elements 11 and 13 are located at the end of each arm of first fluid chamber 14. Pushing elements 11 and 13 may take the form of plungers, formed as flexible diaphragm-s. Alternatively, pushing elements 11 and 13 could be pistons or piezoelectric elements.

At the apex of the ‘V’, separating first fluid chamber 14 from second fluid chamber 16, is a filter 18, having a plurality of apertures 20. Filter 18 may have between 5 and 100 apertures 20. Apertures 20 extend the full height of second fluid chamber 16 and are preferably wide enough so as to allow the analyte particles of interest, if any are present, to pass but to block any particles larger than the analyte particles. For example, for a device 10 to detect HIV, apertures 20 can have a width of between 80 and 150 nm. Various other configurations of apertures will be obvious to a person skilled in the art.

Second fluid chamber 16 has an approximate height of 1100 nm and includes a detection area 17; a mixing channel 23; and a collecting chamber 22. In contact with the interior space of detection area 17 of second fluid chamber 16 are two electrodes 26 and 28. Detection area 17 is in fluid communication with collecting chamber 22 by way of mixing channel 23. Mixing channel 23 is a generally serpentine passageway, approximately 550 nm in width. For simplicity, mixing channel 23 is shown in FIG. 1 with two turns, although it may include more. Collecting chamber 22 is preferably generally circular shaped in order to facilitate mixing of fluids within it. A passageway 24 leads to collecting chamber 22 and enables a reagent solution to be introduced to collecting chamber 22. The reagent solution may initially be contained in a reagent chamber (not shown) in fluid communication with passageway 24. For example, the reagent chamber could be a disposable cartridge. Alternatively, the reagent solution may be added by way of a syringe or otherwise. As with opening 12, passageway 24 is depicted in FIG. 1 as leading from a side of device 10, however, it could alternatively lead from the top of device 10. It may also include a one-way valve to prevent fluid backwash.

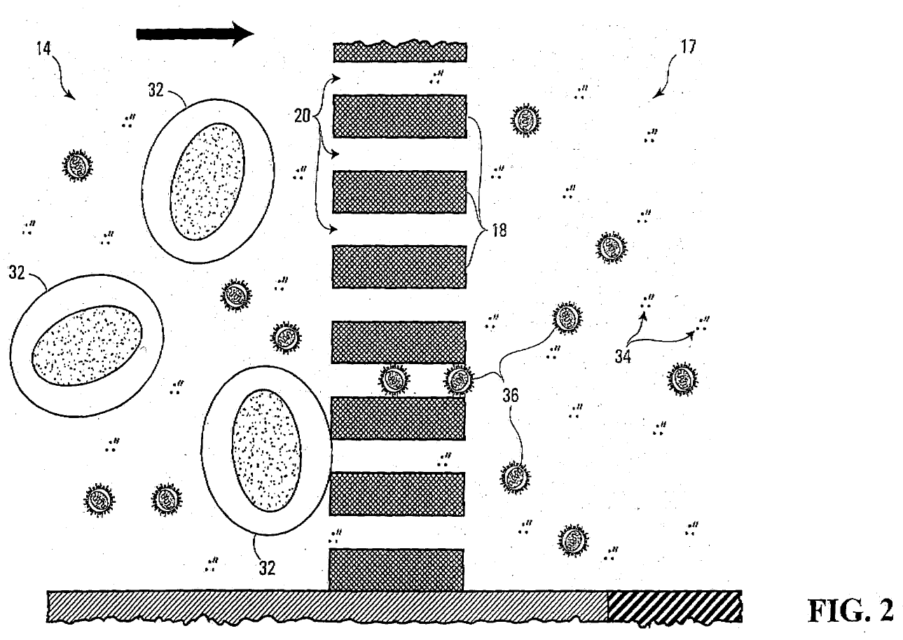

The operation of device 10 can be best described with reference to FIGS. 1-4. A blood sample 30 is introduced to first fluid chamber 14 through opening 12. Blood sample 30 can be of the order of ten microliters in volume and can therefore be provided from a small prick in the finger of a person being tested. Blood sample 30 includes red blood cells 32, white blood cells (not shown) and, other smaller particles 34 such as water, proteins, minerals, and the like. Blood sample 30 may also include analyte particles, the presence of which is to be detected. In the embodiment shown in FIGS. 2-4 the analyte particle to be detected is HIV 36. Optionally, an anti-clotting agent may also be added to first fluid chamber 14 to prevent clotting in blood sample 30.

Once introduced to first fluid chamber 14, blood sample 30 is urged to flow in the directions indicated by the solid-line arrows shown in FIG. 1 by the inward stroke of pushing element 11. Pushing elements 11 and 13 may be moved back and forth in a coordinated fashion, so that the inward stroke of pushing element 11 coincides with the outward stroke of pushing element 13. In this way, the direction of the flow of blood sample 30 in chamber 14 is reversed to flow in the directions indicated by the broken-line arrows shown in FIG. 1 on the inward stroke of pushing element 13. Preferably, the flow of sample 30 in first fluid chamber 14 will be reversed several times. In this way, blood sample 30 flows along the surface of filter 18 repeatedly with a portion of sample 30 flowing transversely and through filter 18 each time.

The flow of sample 30 from first fluid chamber 14 to second fluid chamber 16 may best be described with reference to FIG. 2. Again, apertures 20 of filter 18 are preferably sized so that they allow HIV 36 and smaller particles 34 to pass through filter 18 to second fluid chamber 16 but block the passage of particles larger than HIV 36 such as red blood cells 32 and white blood cells (not shown). By repeatedly reversing the direction of the flow of sample 30 in first fluid chamber 14, the larger particles are discouraged from blocking or clogging apertures 20.

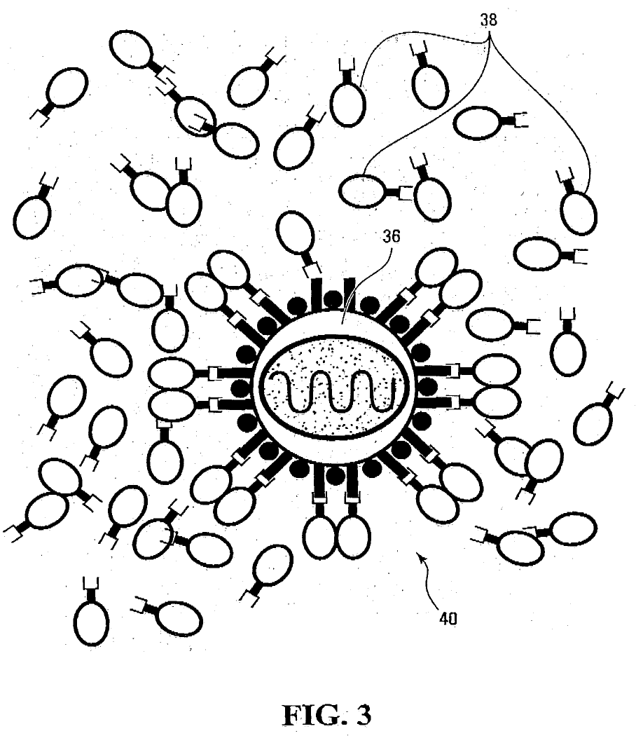

Blood sample 30, including HIV 36, if present, and smaller particles 34, thus flows through filter 18 to second fluid chamber 16. In second fluid chamber 16, blood sample 30 flows from detection area 17, through mixing channel 23 into collecting chamber 22. Once blood sample 30 reaches collecting chamber 22, a reagent solution is added to it. The reagent solution is preferably introduced through passageway 24. The reagent solution contains reagent particles that will react with analyte particles, if any are present, to form reagent-analyte complexes. The reagent particles are smaller in size than the analyte particles, the presence of which is to be detected. For example, the reagent solution can contain truncated CD4 glycoprotein particles 38. CD4 glycoprotein is commonly found in the human body on the surface of white blood cells known as T lymphocytes, or T cells. On the surface of a T cell, CD4 provides a binding site for HIV thus enabling HIV to infect the T cell. A soluble, truncated form of CD4 38, not associated with T cells, will bind with HIV 36 to form a CD4-HIV complex 40. This is depicted in FIG. 3. CD4-HIV complex 40 is larger than both CD4 38 and HIV 36 individually.

Preferably, a suitable amount of 50% (wt/v) CD4 solution is added. For example, enough CD4 may be added to create about a 1 to 1 ratio by volume between the CD4 solution and the pre-filtered blood sample 30. Assuming HIV 36 is present in blood sample 30, some of the HIV 36 and some of the CD4 38 present in collecting chamber 22 will react to form CD4-HIV complexes 40. Mixing, and thus reacting, of HIV 36 and CD4 38 is further encouraged by inducing sample 30, including the CD4 38 that has been added to it, to flow from collecting chamber 22, through mixing channel 23 to detection area 17, i.e. from right to left in second fluid chamber 16 as depicted in FIG. 1. This flow may be, induced by the fluid pressure of the reagent solution entering collecting chamber 22 through passageway 24 or otherwise.

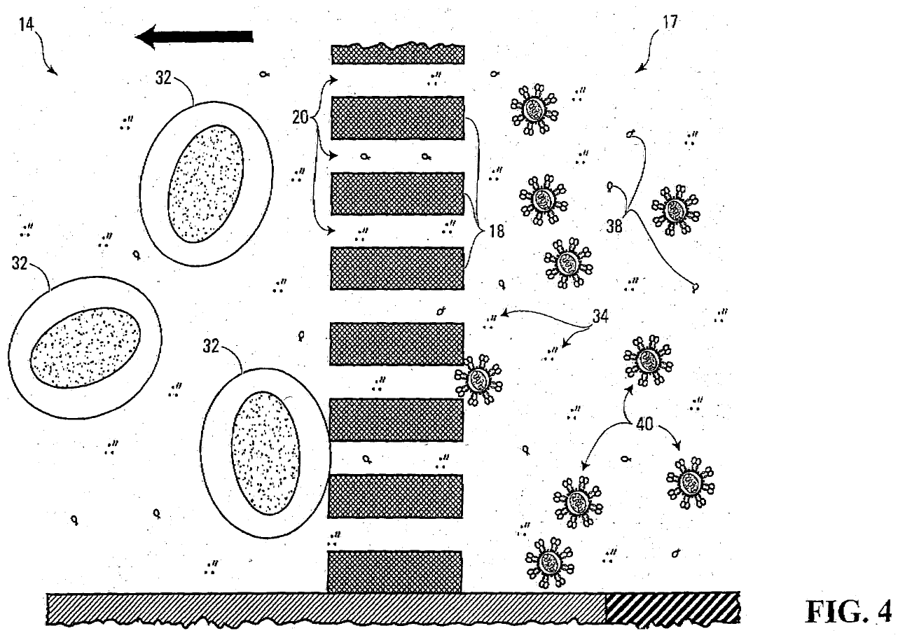

As sample 30 flows through mixing channel 23, more HIV-CD4 reactions occur and the concentration of CD4-HIV complexes 40 rises. As depicted in FIG. 4, when the mixture reaches filter 18, the force of the flow will cause smaller particles 34 and any unreacted CD4 38 and HIV 36 to pass through filter 18 to first fluid chamber 14. As described above, the size of apertures 20 is chosen so as to allow HIV 36 to pass through filter 18 but to block any particles larger than HIV 36. Therefore, because CD4-HIV complexes 40 are larger than simple HIV 36 they will not pass through filter 18 and thus remain in second fluid chamber 16.

Preferably, and to ensure that substantially all unreacted CD4 38 and HIV 36 passes through filter 18 into first fluid chamber 14, after the CD4 solution has been introduced through passageway 24, distilled water may be forced through passageway 24 or introduced into collecting chamber 22 by any other method so that it will flow through device 10 towards first fluid chamber 14.

The presence of CD4-HIV complexes 40 in second fluid chamber 16 can now be detected by a sensing circuit. The sensing circuit includes electrodes 26 and 28, which are in contact with the interior space of detection area 17 of second fluid chamber 16 and a voltage source (not shown), and a suitable conventional electronic circuit capable of detecting and communicating a change in resistivity. CD4-HIV complexes 40 in detection area 17 will affect the resistivity between electrodes 26 and 28. For example, in the absence of other fluids in detection area 17, a low resistivity indicates the presence of CD4-HIV complexes 40, while a high resistivity indicates their absence. The sensing circuit can thus determine this resistivity to detect their presence. A positive test result can be communicated to the user.

It no HIV 36 is present in blood sample 30, no CD4-HIV reactions will occur and no CD4-HIV complexes 40 will be formed. Therefore, the sensing circuit will detect a higher resistivity between electrodes 26 and 28 and a negative test result can be communicated to the user.

Conveniently, device 10 may be disposable. It would thus be suitable for personal home use. Optionally, however, device 10 could be made to be reusable. For example, a cleaning solution, such as 30% (v/v) hydrogen peroxide, could be introduced to device 10 to disinfect its chambers and passageways between uses.

Device 10 may be operated by a user mechanically moving pushing elements 11 and 13 and activating valves (not shown) to release reagent solution, and possibly other fluids, into chambers 14 and 16. Alternatively, the pushing elements and valves of device 10 may be electronically controlled by a processing element (not shown). In this embodiment, pushing elements 11 and 13 could be electronic or electro-mechanical, formed for example as piezoelectric diaphragms. Conveniently, the processing element-may also be used with the sensing circuit to detect the presence of the virus.

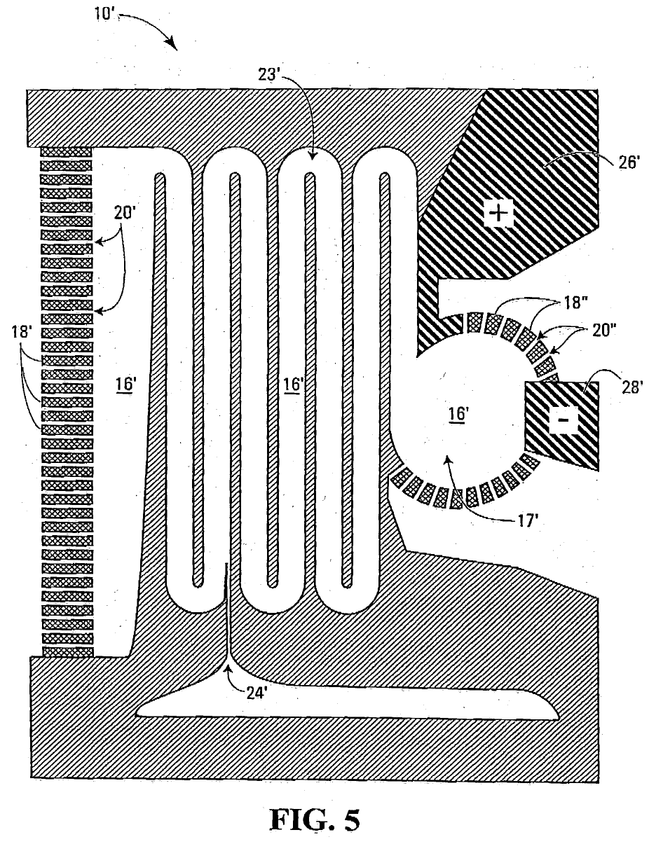

A person of ordinary skill will now appreciate that the present invention could easily be embodied in a variety of configurations. For example, FIG. 5 depicts another possible embodiment of a virus detection device exemplary of the present invention 10′. Device 10′ includes two separate filters 18′ and 18″. For ease of reference, the elements of FIG. 5 are labeled with the same numbers as their corresponding functional counterparts in FIG. 1 but with the prime (′) or double-prime (″) symbol. Device 10′ may also be formed in plastic by micro-injection molding methods or by any other method known in the art. In device 10′ the flow of blood sample 30 is unidirectional, i.e. from left to right in FIG. 5.

Device 10′ includes: a first fluid chamber (not shown); a first filter 18′ having apertures 20′; a second fluid chamber 16′ which includes a serpentine mixing channel 23′ and a detection chamber 17′ which is generally round in shape; a passageway 24′ through which reagent solution is introduced; electrodes 26′ and 28′ which are in contact with the interior of detection chamber 17′, and a second filter 18″ having apertures 20″.

In operation, after blood sample 30 is introduced to a first fluid chamber (not shown) of device 10′, it is urged to flow through first filter 18′. First filter 18′ has apertures 20′ which, like apertures 20 of device 10, are sized to allow analyte particles, HIV 36 in the present example, and smaller particles 34 to pass through but to block any particles larger than the analyte particles. After passing through first filter 18′, sample 30 is then induced to flow through mixing chamber 23′ of second fluid chamber 16′. At a point near the entrance of mixing channel 23′ as depicted in FIG. 5, reagent solution containing CD4 particles 38 is added to sample 30 through passageway 24′ Sample 30, including CD4 particles 38, continues to flow through mixing channel 23′. Assuming HIV 36 is present in sample 30, some of the HIV 36 and C4 38 will react to form CD4-HIV complexes 40.

When sample 30 reaches detection chamber 17′ of second fluid chamber 16′ and second filter 18″, the force of the flow will cause smaller particles 34 and any unreacted CD4 38 and HIV 36 to pass through second filter 18″. Again, the size of apertures 20″ is chosen so as to allow HIV 36 to pass through filter 18″ but to block any particles larger than HIV 36. Therefore, because CD4-HIV complexes 40 are larger than simple HIV 36 they will not pass through second filter 18″ and thus remain in detection chamber 17′ of second fluid chamber 16′.

The presence of CD4-HIV complexes 40 in detection chamber 17′ can now be detected by a sensing circuit that includes electrodes 26′ and 28′ in a manner similar to that described for device 10. Indication of a positive test result can be communicated to the user. If no HIV 36 is present in sample 30, the absence of any CD4-HIV complexes 40 in detection chamber 17′ will be detected and a negative test result will be communicated to the user.

Of course, the above described embodiments are intended to be illustrative only and in no way limiting. The described embodiments of carrying out the invention are susceptible to many modifications of form, arrangement of parts, details and order of operation. The invention, rather, is intended to encompass all such modification within its scope, as defined by the claims.

What is claimed is:

1. A method for detecting the presence of an analyte particle in a fluid, said method comprising, sequentially;
filtering a sample of said fluid to remove particles in said sample larger than said analyte particle;
adding to said sample a reagent that interacts with said analyte particle to form a reagent-analyte particle complex that is larger than said analyte particle;
filtering said sample to remove particles from said sample that are smaller than said reagent-analyte particle complex;
testing said sample for the presence of said reagent-analyte particle complex to detect the presence of said analyte particle in said fluid.

2. A method in accordance with claim 1, wherein said fluid is a biological fluid.

3. A method in accordance with claim 2, wherein said biological fluid is blood.

4. A method in accordance with claim 3, wherein said analyte particle is human immunodeficiency virus.

5. A method in accordance with claim 1, wherein said analyte particle is a virus.

6. A method in accordance with claim 4, wherein said virus is human immunodeficiency virus.

7. A method in accordance with claim 6, wherein said reagent is truncated CD4 glycoprotein.

8. The method of claim 7, wherein said filtering is performed using miro-injected molded plastic.

9. A lab-on-a-chip for detecting the presence of an analyte particle in a fluid, comprising
a first chamber for receiving a sample of said fluid;
a second chamber for receiving a reagent that reacts with said analyte particle in said sample to form a reagent-analyte particle complex, larger than said analyte particle;
a filter in flow communication with said second chamber, said filter sized to pass said analyte particle, and block said reagent-analyte particle complex;
a detector for detecting the presence of said reagent-analyte particle complex in said second chamber.

10. The lab-on-a-chip of claim 9, wherein said filter is in flow communication with said first chamber, to block particles larger than said analyte particle from passing from said first chamber to said second chamber.

11. The lab-on-a-chip of claim 9, further comprising a second filter in flow communication with said first chamber and said second chamber, to block particles larger than said analyte particle from passing from said first chamber to said second chamber.

12. The lab-on-a-chip of claim 9, wherein said fluid is a biological fluid.

13. The lab-on: a-chip of claim 12, wherein said biological fluid is blood.

14. The lab-on-a-chip of claim 9, wherein said analyte particle is a virus.

15. The lab-on-a-chip of claim 14, wherein said virus is human immunodeficiency virus.

16. The lab-n-a-chip of claim 15, wherein said reagent is truncated CD4 glycoprotein.

17. The lab-on-a-chip of claim 9, wherein said second chamber comprises a serpentine mixing passage for mixing said reagent with said analyte particle in said sample.

18. The lab-on-a-chip of claim 9, wherein said detector comprises an electrode for electrically detecting presence of said reagent-analyte particle complex in said second chamber.

19. The lab-on-a-chip of claim 15, wherein said filter is sized to block particles larger than 110 nanometers.

20. The lab-on-a-chip of claim 9, further comprising a pushing element for urging said sample through said filter.

21. The lab-on-a-chip of claim 20, wherein said pushing element is electronically controlled, and further comprising a processor to control said pushing element.
